(19) United States
(12) Patent Application Publication
(10) Pub. No.: US 2007/0156459 A1
(43) Pub. Date: Jul. 5, 2007

(54) System for conducting clinical trials

(75) Inventors: Stephen McMahon, Paddington (AU); Bruce Neal, Bondi (AU)

Correspondence Address: Duane Morris, 1667 K Street NW, Suite 700, Washington, DC, 20006, US

(73) Assignee: GEORGE MEDICA PTY. LTD., NEWTON (AU)

(21) Appl. No.: 10/536,503

(22) PCT Filed: Nov. 27, 2003
(86) PCT No.: PCT/AU03/01582

§ 371 Date: Feb. 8, 2006

(30) Foreign Application Priority Data
Nov. 27, 2002 (AU) 2002952938

(51) Int. Cl. A61B 5/00

(52) U.S. Cl. 705/3

(57) ABSTRACT

There is disclosed a system (1) for conducting a clinical trial of a pharmaceutical substance on a plurality of trial participants. The system (1) includes a central database (2) having a memory means in communication with a database means, one or more trial sites each having a remote computer (3) located remotely from the central database (2) and in communication therewith, the central database (2) being configured to receive predetermined parameters of the trial, both the central database (2) and the remote computers (3) being configured to receive predetermined trial data from one or more trial participants, the central database (2) being configured to control and track the ordering, allocation and dispensing of the pharmaceutical substances and compiling a pharmaceutical substance inventory record on the central database, and the central database (2) being configured to terminate the clinical trial in response to one or more predetermined trial termination conditions. A method of conducting a clinical trial of a pharmaceutical substance on a plurality of trial participants is also disclosed.

The invention relates to a system for conducting clinical trials on people and, in particular, to a system for efficiently conducting clinical trials.

The invention has been developed primarily for use with the clinical trials of pharmaceutical substances and devices and methods of treatment and will be described hereinafter with reference to these applications. However, it will be appreciated that the invention is not limited to this particular field of use.

As part of the process of introducing pharmaceutical substances, devices or methods to treat viruses, diseases or other undesirable conditions in people, several phases of trial must be conducted to ensure that the ultimate end user of such a pharmaceutical, device or method receives not only a benefit from the pharmaceutical substance but also does not suffer from any undesirable side-effects.

Pharmaceutical substances, devices or methods of treatment are often tested on animals as an initial step to assess their effectiveness and also whether any undesirable side effects will accompany their use.

Once the testing on animals has been completed, the results of the test are reviewed and if the test results satisfy some predetermined conditions, then clinical trials of the pharmaceutical, device or method may be conducted on humans to further evaluate and possibly refine the device, method or pharmaceutical substance or its dosage.

As would be expected, the clinical trials on people must be conducted under procedures determined by local authorities, for example the Food and Drug Administration in the US and the Therapeutic Goods Administration in Australia.

As is commonly known in most countries, the regulatory requirements for having a pharmaceutical substance, device or method of treatment available to people generally requires rigorous testing and reporting procedures to be followed to satisfy local authorities and for the ultimate safety of the consumer. These required trials and reports are therefore critical to the ultimate commercial success of the pharmaceutical product or device of method of treatment.

In the case of pharmaceutical substances, it is also commonly known that conducting clinical trials on people is a long process at least due to the fact that unwanted side effects related to the pharmaceutical or a formulation may appear a substantial period of time after the pharmaceutical product was trialed. Similarly, the dosage level and rate and efficiency of the pharmaceutical substance in treating a condition generally also take a significant period of time to assess. Similarly, in the case of devices such as implants or methods of treatment, any unwanted side effects may not appear for a substantial time.

Many different professionals or technicians are involved in such a clinical trial of the pharmaceutical substance, device or method of treatment and it has been found that information related to such a trial can be scattered over a variety of institutions or stored in a not easily accessible manner to those monitoring the trial, for example. This ultimately prolongs the trial process with the primary effect of incurring additional expense by keeping the pharmaceutical, device or method of treatment from the market.

An important aspect of the conduct of clinical trials of a pharmaceutical substance relates to its manufacture, distribution, consumption and disposal. Many known methods of conducting clinical trials do not have a pharmaceutical substance inventory monitoring capability which, in addition to creating difficulties for the trial workers to monitor the pharmaceutical substances, unnecessarily slows the overall progress of a clinical trial and makes accounting for all of the substance difficult. Similarly in the case of devices and methods of treatment, a lack of monitoring in known methods of conducting clinical trials disadvantageously provides difficulties in monitoring the devices and any equipment associated with the methods of treatment.

Furthermore, in the known methods of conducting clinical trials, relatively large volumes of printed matter relating to the trial are produced. It is known that the tracking of all of the printed matter is difficult and provides multiple access points for corrupt data to be included in the trial. US patent publication no. 2002/0165875 (Verta) provides a data tabulation and correlation system. The system is limited to preparing data for presentation, however, it fails to address the deficiencies of the prior art as above. For example, paragraph [0016] states that the advantage of the system of this publication resides in presenting data obtained in clinical trials. Similarly in US 2002/0120573 (McCormick) which provides a system for controlling the distribution of controlled information to health care professionals. The system of this publication is principally aimed at protecting privacy from marketing by authenticating identification of the physician electronically.

Although not addressing the above deficiencies of the prior art, U.S. Pat. No. 6,196,970 (Brown) provides a method and system of recording data in clinical trials in which ambiguity of the recorded data is minimized. In language more consistent with the publication, “fuzziness” inherently included in subjective answers is minimized.

In WO 02/051354 (Becker) there is provided a system that aims to provide objective criteria to support the continuation or discontinuation of medical intervention in either general practice or clinical trial patients. The system uses a complex decision algorithm that may improve the efficiency and safety of drug treatments.

In US patent publication No. 2002/0023083 (Durkalski) there is provided a modular system to allow untrained or unskilled users to build clinical trial data management systems, similarly aimed to the disclosure of the VERTA publication above. Unfortunately, this system provides a data management and entry system that doesn't address the deficiencies of the prior art.

It is an object of the invention to provide a system for conducting clinical trials which will overcome or substantially ameliorate at least some of these deficiencies of the prior art or provide a useful alternative.

According to a first aspect of the invention there is provided a method of conducting a clinical trial of a device or method or substance of treatment on a plurality of trial participants, the method including the steps of:

establishing an electronic database in communication with one or more remote computers;

entering predetermined trial parameters of the conduct of the clinical trial into the database;

programming the database and remote computers to provide a predetermined interface for accepting predetermined information relating to the trial being entered by trial participants, administrators and/or auditors;

recording particulars of the trial participants and forming ordered registration information on the database;

forming randomised particulars of the trial participants in the database from the ordered registration information, the randomised particulars including the allocation of an identifier label,

assigning the device or method or substance of treatment to the randomised particulars of each trial participant;

entering trial data via the predetermined interface into the database by an authorized trial participant;

producing a report of data entered onto the database in response to predetermined reporting conditions;

controlling and tracking the ordering, allocation and dispensing of the device or method or substance of treatment and compiling a method or substance inventory record on the database; and

terminating the clinical trial in response to predetermined termination conditions.

Preferably, the method is for conducting a clinical trial of a pharmaceutical substance and the database and remote computers communicate via internet communications.

In preferred embodiments, the predetermined trial parameters include the dosage rates of the pharmaceutical substance to be given to the selected trial participants. More preferably, the trial data is entered onto the remote computer or the database and wherein only specific volumes and forms of the data are acceptable by the remote computer or central database.

Preferably, the trial administrators have access to view any entered data or add any predetermined data to the information in the database, and the trial auditors have access to view any entered information in the database and the recorded particulars of the selected participants in the ordered registration information are restricted to predetermined trial administrators and auditors.

In preferred embodiments, the randomised particulars of the selected trial participants and trial information relating to those participants are available to all trial participants.

Preferably, the method includes the step of generating reminders from the database at predetermined times after trial data is entered, the reminders being displayed to predetermined trial participants upon access to the remote computers or the database. More preferably, the trial report of entered data reports on all data entered into the database at a predetermined time or in response to the entry of specific data types or quantities.

In preferred embodiments, the step of controlling and tracking the movement of the pharmaceutical substances and recording the pharmaceutical substance inventory record on the database further includes the step of selectively establishing communication with the pharmaceutical substance supplier and placing an electronic order. More preferably, the method includes the steps of:

providing one or more local trial administration centres for conducting the clinical trial;

assigning one or more trial participants to each local trial administration centre;

determining a payment to each local trial administration centre for conducting the clinical trial; and

effecting the determined payment to each local trial administration centre at predetermined times from the commencement of the clinical trial. Yet more preferably, the determined payments are determined in response to types of treatment delivered to trial participants and a standard amount per patient per clinical trial visit.

Preferably, the method includes the step of providing financial reports relating to the determined payments including payments earned by the local trial administration centres, payments made thereto, payments outstanding to each local trial administration centre, and over-payments previously made to any local trial administration centre.

In preferred embodiments, the trial termination conditions include a lapsing of a predetermined time, consumption of a predetermined amount of pharmaceutical substance by one or more trial participants, or the occurrence of an adverse event of a trial participant.

Preferably, the method includes a plurality of remote computers each being disposed at individual sites remote from the database and being configured to accepted on predetermined data and the remote computers are selected from the group consisting of: personal digital assistants, laptop computers, desktop computers, tablet personal computers, mobile telephones, pagers and dedicated computing devices. More preferably, the remote computers and electronic database communicate by wireless, electrical cable and/or optical fibre networks and the electronic database includes a computer server in combination with a data storage device.

More preferably, a plurality of pharmaceutical substances are simultaneously trialed and controlled by the database.

According to another aspect of the invention there is provided a system for conducting a clinical trial of a device or method or substance of treatment on a plurality of trial participants, the system including:

a database having a memory means in communication with a database means;

one or more trial sites each having a remote computer located remotely from the database and in communication therewith;

the database being configured to receive predetermined parameters of the trial;

both the database and the remote computers being configured to receive predetermined trial data from one or more trial participants; and

the database being configured to control and track the ordering, allocation and dispensing of the device or method or substance of treatment and compiling a device or method or substance inventory record on the central database;

wherein the database being configured to terminate the clinical trial in response to one or more predetermined trial termination conditions.

In preferred embodiments, the system is configured for conducting a clinical trial of a pharmaceutical substance and the database is configured to receive and record information relating to the trial participants and also to form randomised particulars of the trial participants in the database including the determination of which trial participants receive the pharmaceutical substance and which receive a placebo.

Preferably, the database is configured to produce a report of data entered into the database relating to the trial. More preferably, the database is configured to generate reminders to the trial administrators at a predetermined time after trial data is entered or the trial commenced, the reminders being displayed upon the trial administrators accessing a remote computer. Yet more preferably, the remote computers are selected from the group consisting of: personal digital assistants, laptop computers, desktop computers, tablet personal computers, mobile telephones, pagers and dedicated computing devices and the remote computers and database communicate by wireless, electrical cable and/or optical fibre networks.

In preferred embodiments, the system is configured to provide one or more local trial administration centres for conducting the clinical trial;

assign one or more trial participants to each local trial administration centre;

determine a payment to each local trial administration centre for conducting the clinical trial; and

effect the determined payment to each local trial administration centre at predetermined times from the commencement of the clinical trial.

Preferably, the determined payments are determined in response to types of treatment delivered to trial participants and a standard amount per patient per clinical trial visit. More preferably, the system is configured to provide financial reports relating to the determined payments including payments earned by the local trial administration centres, payments made thereto, payments outstanding to each local trial administration centre, and over-payments previously made to any local trial administration centre.

A preferred embodiment will now be described, by way of example only, with reference to the accompanying drawing in which:

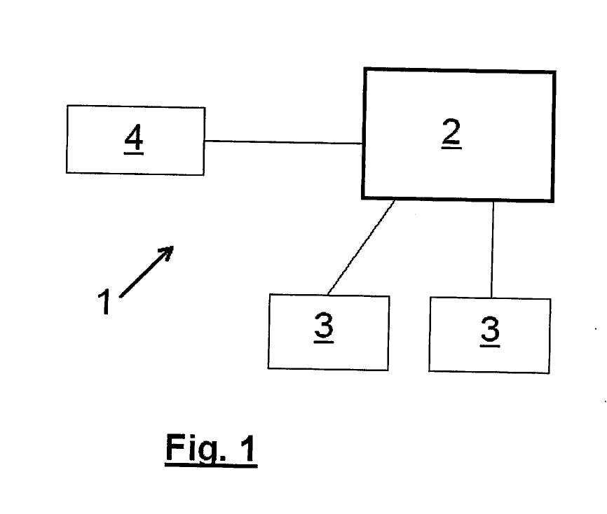

FIG. 1 is a schematic view of a system in accordance with the embodiment.

Referring to the drawing, there is illustrated a system 1 of conducting a clinical trial of a pharmaceutical substance or device or method of treatment on a plurality of trial participants and the system 1 will be described with reference conducting a clinical trial of a pharmaceutical substance. The trial participants are located over a plurality of study sites. However, in other embodiments not illustrated, the trial participants are located at one site.

An electronic database 2 is disposed at one location and a remote computer 3 in communication with the database 2 is disposed at each of the plurality of remote study sites. The remote computer 3 can be in the form of a personal digital assistant (PDA), laptop or other personal computer, mobile telephone or other device. The communication link between the remote computer 3 and the database 2 can communicate by any conventional cable or wireless communication means. It is also noted that the electronic database 2 can include any processor in combination with a memory means such as a computer server in combination with a data storage device or a plurality of processors and/or memory means or any other conventional database arrangement.

The parameters by which the clinical trial is to be conducted are entered into the database 2. These trial parameters or rules must comply with local regulations including the timing of the trial and definitions of functions of the remote trial sites as well as any dosage rates of the pharmaceutical substance being trialed.

Both of the database 2 and remote computers 3 are programmed to provide a predetermined interface to a user. In this interface, the database or remote computers will only accept predetermined information in a predetermined form to standardise data collection from trial participants and trial administrators. For example, dates relating to events in the clinical trial must be recorded in the format MM/DD/YY. Further, the interface to the database 2 and remote computers 3 is configured to accept identifying information and medical history of trial participants.

Once the total number of trial participants has been finalised, their personal particulars are entered onto the database and/or remote computers to an ordered registration information file. The information in the ordered registration information files is available to predetermined trial administrators and trial auditors and any other predetermined persons. It is noted that the trial auditors can be any relevant party including overseeing doctors or government bodies for example.

The database 2 then forms a randomised particulars file on the database 2 for each trial participant in the ordered registration information file. The randomised particulars files are formed once the ordered registration information files are created. However, in other embodiments, the randomised particulars files are selectively created in batches.

The randomised particulars file includes the allocation of a label for identifying the trial participant. Furthermore, the database 2 randomly selects which trial participants receive the pharmaceutical substance as part of the clinical trial and which trial participants receive a placebo. This information is recorded in only the ordered registration information files and only predetermined auditors, for example, can correlate the randomised particulars files with corresponding ordered registration information files. The allocation of the pharmaceutical can also be performed after the randomised parts files are generated, or as part of the process.

In the conduct of the clinical trial, data relating to each trial participant as well as any other information relevant to the assessment of the clinical trial is entered via the predetermined interface into the database 2 by either a trial participant or trial administrator (authorised trial participants). Generally, the data will be entered by a trial administrator. The data can be entered via remote computers 3 and communicated to the database 2. When a trial administrator enters data relating to a trial participant, it must be in a predetermined format, for example, a predetermined combination of alphanumeric characters in order to be accepted by the remote computers 3 or database 2. It is noted that the trial administrator can be a combination of remote locally located trial administrators where the clinical trials are conducted.

The database 2 is configured to produce a report of trial activity at predetermined times from the commencement of the clinical trial. These reports can be used by the trial administrators to review the conduct of the trial as well as by auditors to ensure that correct procedures are being followed. Importantly, the reports can also include details of any adverse events including the trial. The reports can also be generated in response to a specific query by a trial administrator or trial auditor relating to a specific trial participant or participants or, for example, a report based on the total number of trial participants receiving a particular pharmaceutical substance dose rate. Therefore, reports including any predetermined information can be generated.

The reports are also generated in response to data entered into the database 2 meeting predetermined conditions such that a ruling or adjudication of the entered trial results needs to occur. That is, entered data meeting predetermined conditions triggers a request for the trial administrators to adjudicate the results.

The database 2 also controls the allocation of the pharmaceutical substance being trialed to the trial participants. The database 2 orders a predetermined quantity of the pharmaceutical substance depending on the number of trial participants and their dosages. The database 2 monitors the use of the pharmaceutical substance from data entered during the trial and compares this information against anticipated pharmaceutical substance usage rates. A record of the pharmaceutical substance is maintained by the database 2 in a pharmaceutical substances inventory record.

Once the quantities of pharmaceutical substance for the trial participants reach a lower threshold level, the database 2 automatically establishes communication with the supplier 4 of the pharmaceutical substances and re-orders a predetermined quantity depending on the future needs of the trial. The control of the ordering, allocation and dispensing of pharmaceuticals or devices being trailed is critical in situations where the pharmaceuticals or devices are not readily obtainable otherwise and require a lead time for manufacture, or in situations where the pharmaceuticals or devices may be harmful if used by parties unrelated to the trial.

The database 2 further monitors the trial data entered and terminates the trial in response to one or more predetermined termination conditions. The predetermined termination conditions include the elapsing of a predetermined time for the trial, the consumption of a predetermined amount of pharmaceutical substance by one or more of the trial participants, or the occurrence of an adverse event in a trial participant. Such adverse events include unpredicted side-effects or other illnesses. Database 2 can also terminate the trial in a number of trial participants or in the trial as a whole in response to a termination condition.

The database 2 and the remote computers 3 communicate over the internet. The database 2 and computers 3 each include a modem for allowing communication between the database 2 and computers 3 to be achieved. In other embodiments of the invention not illustrated, the database 2 and computers 3 each include a network card for allowing dedicated communications therebetween. Similarly in the case of the database 2 establishing communication with the supplier of the pharmaceutical substance, the supplier 4 and database 2 communicate via a modem or dedicated network link. Alternatively, the database 2 and computer 3 can communicate by wireless means.

In respect of accessibility to the ordered registration information files containing information relating to each trial participant and data entered throughout the trial, only predetermined trial administrators and trial auditors can read the data and no person can erase or re-write data. The database 2 records a log of access to trial information, for example an ordered registration information file, primarily to avoid the possibility of the data being deleted or altered by unauthorised persons. In this way, the integrity of the data is maintained by not allowing a means of falsifying data.

However, the randomised particulars files are able to be read by trial administrators and auditors, but not trial participants. It is noted that the randomised particulars files do not identify the participant directly or whether they are consuming the pharmaceutical substance or placebo as part of the trial.

The system 1 also includes a reminder generator, not illustrated, integrated into the database 2. When data is entered into the database 2, it will trigger the generation of a reminder following up the data at a predetermined advance date. The advance date depends on the data entered, for example, when an ordered information file is created, a reminder is generated for one week hence to ensure that a corresponding randomised particulars file has also been created. Similarly, when an adverse event of a trial participant or participants is entered into the database 2, a reminder is automatically generated for an advance date to follow up the entry of the adverse event. However, the system 1 can be configured to generate any other required reminders, for example, based on an elapsed time or the occurrence of a predetermined event.

Although not illustrated, the system 1 is configured to provide one or more local trial administration centres for conducting the clinical trial. That is, one or more local trial administration centres are used and assign one or more trial participants to each local trial administration centre depending, for example, on the trial participant locality or type of treatment provided in the clinical trial.

The system 1 then determines a payment to be made to each local trial administration centre for conducting the clinical trial on the assigned trial participants.

Once the payment has been determined by system 1, the determined payment to each local trial administration centre is effected (or paid) at predetermined times from the commencement of the clinical trial. Generally, the predetermined times at which the payment is made are selected by the trial administrator. In the embodiment of FIG. 1, the system effects payment to each local trial administration centre at the time each centre becomes a party to the clinical trial and every six months thereafter, and in response to predetermined data being entered by the authorised trial participants. That is, determined payments occur at the predetermined times and only if data corresponding to the completion of clinical trial visits entered by the trial administrator.

The determined payments are determined in response to types of treatment delivered to trial participants and a standard amount per patient per clinical trial visit. For example, for a trial participant receiving a placebo they will receive a lower determined payment than if the trial participant receives a pharmaceutical or medical device. Similarly, where the local trial administrators visit a participant at the participant's home, call them on the telephone or provide another form of contact, different payment amounts will be determined.

The system 1 is configured to provide financial reports relating to the determined payments including payments earned by the local trial administration centres, payments made thereto, payments outstanding to each local trial administration centre, and over-payments previously made to any local trial administration centre. The financial reports can be created at predetermined times or in response payments being made or other condition.

It is also noted that the system 1 can be used to trial more than one pharmaceutical substance simultaneously. For example, the trial parameters can be predefined to include a trial of an additional pharmaceutical substance.

Likewise, the system 1 can be used to trial one pharmaceutical substance, however, variants or different formulations of the substance can also be simultaneously trialed within the one trial. The variants of a pharmaceutical substance can include biochemical variants or variants in physical state such as liquid or tablet. For example, some participants in a pharmaceutical substance trial can be given a liquid form of the substance, other participants given a tablet form of the substance and yet other participants given biochemically varied forms of the substance in either liquid or tablet form.

The database 2 and remote computers 3 are also configured to selectively access information relating to the trial via the internet. For example, the database 2 and remote computers 3 can access the local regulatory framework for conducting the trials.

It will be appreciated that although the foregoing describes a preferred embodiment of the invention relating to a method and system for conducting clinical trials of a pharmaceutical substance, it will be appreciated that the method and system are equally applicable to the conduct of clinical trials of devices or methods of treatment or surgical techniques, for example. That is, the foregoing describes only a preferred embodiment of the present invention and modifications, obvious to those skilled in the art, can be made thereto without departing from the scope of the present invention.

1. A method of conducting a clinical trial of a device or method or substance of treatment on a plurality of trial participants, the method including the steps of:
establishing an electronic database in communication with one or more remote computers;
entering predetermined trial parameters of the conduct of the clinical trial into the database;
programming the database and remote computers to provide a predetermined interface for accepting predetermined information relating to the trial being entered by trial participants, administrators and/or auditors;
recording particulars of the trial participants and forming ordered registration information on the database;
forming randomised particulars of the trial participants in the database from the ordered registration information, the randomised particulars including the allocation of an identifier label;
assigning the device or method or substance of treatment to the randomised particulars of each trial participant;
entering trial data via the predetermined interface into the database by an authorized trial participant;
producing a report of data entered onto the database in response to predetermined reporting conditions;
controlling and tracking the ordering, allocation and dispensing of the device or method or substance of treatment and compiling a method or substance inventory record on the database; and
terminating the clinical trial in response to predetermined termination conditions.

2. A method according to claim 1 wherein the method is for conducting a clinical trial of a pharmaceutical substance.

3. A method according to claim 1 wherein the database and remote computers communicate via internet communications.

4. A method according to claim 2 wherein the predetermined trial parameters include the dosage rates of the pharmaceutical substance to be given to the selected trial participants.

5. A method according to claim 1 wherein the trial data is entered onto the remote computer or the database and wherein only specific volumes and forms of the data are acceptable by the remote computer or central database.

6. A method according to claim 1 wherein the trial administrators have access to view any entered data or add any predetermined data to the information in the database, and the trial auditors have access to view any entered information in the database.

7. A method according to claim 1 wherein the recorded particulars of the selected participants in the ordered registration information are restricted to predetermined trial administrators and auditors.

8. A method according to claim 1 wherein the randomised particulars of the selected trial participants and trial information relating to those participants are available to all trial participants.

9. A method according to claim 1 including the step of generating reminders from the database at predetermined times after trial data is entered, the reminders being displayed to predetermined trial participants upon access to the remote computers or the database.

10. A method according to claim 1 wherein the trial report of entered data reports on all data entered into the database at a predetermined time or in response to the entry of specific data types or quantities.

11. A method according to claim 2 wherein the step of controlling and tracking the movement of the pharmaceutical substances and recording the pharmaceutical substance inventory record on the database further includes the step of selectively establishing communication with the pharmaceutical substance supplier and placing an electronic order.

12. A method according to claim 1 including the steps of:
providing one or more local trial administration centres for conducting the clinical trial;
assigning one or more trial participants to each local trial administration centre;
determining a payment to each local trial administration centre for conducting the clinical trial; and
effecting the determined payment to each local trial administration centre at predetermined times from the commencement of the clinical trial.

13. A method according to claim 12 wherein the determined payments are determined in response to types of treatment delivered to trial participants and a standard amount per patient per clinical trial visit.

14. A method according to claim 12 including the step of providing financial reports relating to the determined payments including payments earned by the local trial administration centres, payments made thereto, payments outstanding to each local trial administration centre, and over-payments previously made to any local trial administration centre.

15. A method according to claim 2 wherein the trial termination conditions include a lapsing of a predetermined time, consumption of a predetermined amount of pharmaceutical substance by one or more trial participants, or the occurrence of an adverse event of a trial participant.

16. A method according to claim 1 including a plurality of remote computers each being disposed at individual sites remote from the database and being configured to accepted on predetermined data.

17. A method according to claim 2 wherein a plurality of pharmaceutical substances are simultaneously trialed and controlled by the database.

18. A method according to claim 1 wherein the remote computers are selected from the group consisting of: personal digital assistants, laptop computers, desktop computers, tablet personal computers, mobile telephones, pagers and dedicated computing devices.

19. A method according to claim 1 wherein the remote computers and electronic database communicate by wireless, electrical cable and/or optical fibre networks.

20. A method according to claim 1 wherein the electronic database includes a computer server in combination with a data storage device.

21. A system for conducting a clinical trial of a device or method or substance of treatment on a plurality of trial participants, the system including:
a database having a memory means in communication with a database means;
one or more trial sites each having a remote computer located remotely from the database and in communication therewith;
the database being configured to receive predetermined parameters of the trial;
both the database and the remote computers being configured to receive predetermined trial data from one or more trial participants; and
the database being configured to control and track the ordering, allocation and dispensing of the device or method or substance of treatment and compiling a device or method or substance inventory record on the central database;
wherein the database being configured to terminate the clinical trial in response to one or more predetermined trial termination conditions.

22. A system according to claim 21 configured for conducting a clinical trial of a pharmaceutical substance.

23. A system according to claim 22 wherein the database is configured to receive and record information relating to the trial participants and also to form randomised particulars of the trial participants in the database including the determination of which trial participants receive the pharmaceutical substance and which receive a placebo.

24. A system according to claim 21 wherein the database is configured to produce a report of data entered into the database relating to the trial.

25. A system according to claim 21 wherein the database is configured to generate reminders to the trial administrators at a predetermined time after trial data is entered or the trial commenced, the reminders being displayed upon the trial administrators accessing a remote computer.

26. A system according to claim 21 wherein the remote computers are selected from the group consisting of: personal digital assistants, laptop computers, desktop computers, tablet personal computers, mobile telephones, pagers and dedicated computing devices.

27. A system according to claim 21 wherein the remote computers and database communicate by wireless, electrical cable and/or optical fibre networks.

28. A system according to claim 21 wherein the database includes a computer server in combination with a data storage device.

29. A system according to claim 21 being configured to:
provide one or more local trial administration centres for conducting the clinical trial;
assign one or more trial participants to each local trial administration centre;
determine a payment to each local trial administration centre for conducting the clinical trial; and
effect the determined payment to each local trial administration centre at predetermined times from the commencement of the clinical trial.

30. A system according to claim 29 wherein the determined payments are determined in response to types of treatment delivered to trial participants and a standard amount per patient per clinical trial visit.

31. A system according to claim 29 configured to provide financial reports relating to the determined payments including payments earned by the local trial administration centres, payments made thereto, payments outstanding to each local trial administration centre, and over-payments previously made to any local trial administration centre.
